(19) United States
(12) Patent Application Publication
(10) Pub. No.: US 2023/0330011 A1
(43) Pub. Date: Oct. 19, 2023

(54) GUMMY COMPOSITION AND METHODS OF USE THEREOF

(71) Applicant: Better Nutritionals LLC, Norco, CA (US)

(72) Inventors: Yundi Liang, Gardena, CA (US); Roger Arthur Tyre, Norco, CA (US)

(21) Appl. No.: 17/977,302

(22) Filed: Oct. 31, 2022

Related U.S. Application Data
(60) Provisional application No. 63/244,134, filed on Sep. 14, 2021.

(51) Int. Cl. A61K 9/00; A61K 36/73; A61K 31/4045; A61K 31/522; A61K 36/185; A61K 31/00; A61K 31/167; A61K 31/192; A61K 31/01; A61K 31/4188; A61K 38/39; A61K 47/36; A61K 47/26; A23G 3/42; A23G 3/36; A23G 3/48; A23G 3/44

(52) U.S. Cl. CPC A61K 9/0056; A61K 36/73; A61K 31/4045; A61K 31/522; A61K 36/185; A61K 31/658; A61K 31/167; A61K 31/192; A61K 31/01; A61K 31/4188; A61K 38/39; A61K 47/36; A61K 47/26; A23G 3/42; A23G 3/362; A23G 3/48; A23G 3/368; A23G 3/44

(57) ABSTRACT

The present technology provides composition that includes pectin and a rare sugar, polydextrose, or a combination thereof; wherein the composition is a gummy and essentially free of gelatin. The composition has less than about 50 wt % sugar, a glycemic index of about 50 or less, about 10 calories or less, or a combination of two or more thereof. The gummy can be used to treat a patient such a patient with a nutritional deficiency or preventing a nutritional deficiency.

### CROSS-REFERENCE TO RELATED APPLICATIONS

This application claims priority to U.S. Provisional Application No. 63/244,134 filed Sep. 14, 2021.

### FIELD

The present technology relates generally to the field of edible gummy compositions comprising a vitamin, mineral, green blend, vegetable blend, fruit blend, probiotic culture, antioxidant, fiber blend, active ingredient, or a combination of two or more thereof.

### BACKGROUND

Current gummy confectioneries such as gummy vitamins are commonly made with a high weight percent of simple sugars (e.g., sucrose and/or glucose). These sugars have a high glycemic index (GI) value, which upon consumption can lead to a spike in glucose levels. Sugars with a high GI value are not tolerated well by people with metabolic disorders such as diabetes.

### SUMMARY OF THE INVENTION

The present technology provides a composition that includes pectin; and a rare sugar, polydextrose, or a combination thereof; wherein the composition is a gummy, essentially free of gelatin, and has less than about 50 wt % sugar.

The present technology also provides a composition that includes pectin; and a rare sugar, polydextrose, or a combination thereof; wherein the composition is a gummy, essentially free of gelatin, and has a glycemic index of about 50 or less.

In another aspect, the present technology also provides a composition that includes pectin; and a rare sugar, polydextrose, or a combination thereof; wherein the composition is a gummy, essentially free of gelatin, and has about 10 calories or less.

The composition can include the polydextrose and the rare sugar (e.g., allulose, tagatose, sorbose, apiose, allose, altrose, and/or melezitose). The composition can also include a vitamin, mineral, green blend, vegetable blend, fruit blend, probiotic culture, antioxidant, and/or fiber blend.

The present technology also provides a method for treating a patient with a nutritional deficiency or preventing a nutritional deficiency, the method including administering at least one gummy composition as disclosed herein.

### DETAILED DESCRIPTION

The definitions of certain terms as used in this specification are provided below. Unless defined otherwise, all technical and scientific terms used herein generally have the same meaning as commonly understood by one of ordinary skill in the art to which the present technology belongs.

The following terms are used throughout as defined below.

As used herein and in the appended claims, singular articles such as “a”, “an”, and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

About in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

Sugar refers to sweet-tasting, soluble carbohydrates commonly used in food. Sugars include hexose monosaccharides, disaccharides, and trisaccharides. Examples of sugar include sucrose (i.e., table sugar), dextrose, glucose, fructose, galactose, mannose, levulose, lactose, maltose, syrup, honey, molasses, treacle, and sugars from concentrated fruit or vegetable juices. Sugar can intend to exclude rare sugars and sugar alcohols.

Monosaccharide refers to to the simplest form of sugar and the most basic units (monomers) of carbohydrates. The general formula is CnH₂nOn, or [Cn(RO)n] or (CH₂0)n. Examples of monosaccharides that are not rare sugars include, but not limited to, glucose (dextrose), fructose (levulose), and galactose. Monosaccharides are the building blocks of disaccharides and polysaccharides.

Disaccharide refers to to the sugar formed when two monosaccharides are joined by glycosidic linkage. Examples of disaccharides include, but are not limited to, sucrose, lactose, maltose, trehalose, cellobiose, chitobiose, kojibiose, nigerose, isomaltose, sophorose, laminaribiose, gentiobiose, trehalulose, turanose, maltulose, leucrose, isomaltulose, gentiobiulose, mannobiose, melibiose, melibiulose, rutinose, rutinulose, and xylobiose.

Oligosaccharide refers to to a sugar polymer containing a small number of monosaccharides (e.g., three to ten). Examples of polysaccharides include, but are not limited to, maltodextrins, cellodextrins, affinose, maitotriose, umbelliferose, raffinose, planteose, sialyla(2-3)lactose, stachyose, lychnose, isolychnose, sesarnose, verbascose, lacto-N-fucopentaose, and oligofructose.

Syrup refers to to glucose syrup, corn syrup, maple syrup, high fructose corn syrup, golden syrup, can syrup, and agave syrup.

Rare sugar refers to a sugar that occurs in limited quantities in nature. Rare sugars can be produced commercially using microbial enzymes. Rare sugars include hexose monosaccharides and derivatives thereof. The rare sugar can be hydrolyzed or partially hydrolyzed disaccharides and/or trisaccharides. The rare sugar can be vegan. The rare sugar can exhibit a limited impact on increasing blood sugar. The rare sugar can have a low glycemic index.

Sugar alcohol refers to organic compounds derived from sugars that contain one hydroxyl group attached to each carbon atom. Examples of sugar alcohols include arabitol, erythritol, glycerol, isomalt, lactitol, maltitol, mannitol, xylitol, sorbitol, or a combination of two or more thereof.

Natural sugar refers to sugar substitute that comes from a plant. Natural sugars include yacon syrup, oligofructose, inulin, brazzein, curculin, glycyrrhizin, luo han guo, miraculin, monellin, pentadin, stevia, thaumatin, or a combination of two or more thereof.

Artificial sweetener refers to synthetic sugar substitutes. For example, alitame, neotame, saccharin, cyclamate, aspartame, sucralose, acesulfame, or a combination of two or more thereof.

High potency or high intensity sweetener refers to a sweetener that is sweeter than sucrose, but contributes minimal or no calories when added to foodstuff. High potency or high intensity sweeteners can be at least about 2-times sweeter than sucrose. High potency or high intensity sweeteners can be at about 10-times, at least about 25-times, at about 50-times, at least about 75-times, at least about 100-times, about 125-times, at least about 150-times, at about 175-times, or at least about 200-times sweeter than sucrose. Natural sugars and artificial sweeteners can also be high potency sweeteners. Exemplary high potency sweeteners include those derived from Stevia (including S. eupatoria, S. ovata, S plummerae, S. rebaudiana, S. salicifolia, S. serrata, and/or S. rebaudiana), saccharin, aspartame, acesulfame potassium, sucralose, neotame, advantame, those derived from Siraitia grosvenorii, or a combination of two or more thereof.

Pectin refers to a gelling agent that is a structural acidic heteropolysaccharide contained in the primary and middle lamella and cells walls of land grown plants. Its main component is galacturonic acid. Pectin can be methoxyl, low methoyl, and/or amidated.

Gum refers to a water-soluble polysaccharide. Non-limiting examples of the gum include, but are not limited to, guar gum, gum acacia, cellulose gum, tragacanth gum, locust bean gum, karaya gum, ghatti gum, tara gum, konjac gum, xanthan gum, curdlan gum, gellan gum, algin gum, and carrageenan gum.

A subject or patient refers to a mammal, such as a cat, dog, rodent or primate. Typically the subject is a human. For example, a human suffering from nutritional deficiency or at risk of suffering from a nutritional deficiency capable of treatment with an effective amount of the compositions of the present technology. Subject and patient can be used interchangeably.

Administering or the administration of a composition to a subject refers to orally introducing or delivering to a subject an effective amount of a composition of the present technology to perform its intended function. Administration includes self-administration and the administration by another.

Treating or treatment refers to alleviation, in whole or in part, of symptoms. For example, the symptoms of a vitamin deficiency, associated with a disorder or disease, or slowing, inhibition or halting of further progression or worsening of those symptoms, or prevention or prophylaxis of the disease or disorder in a subject at risk for developing the disease or disorder. Preventing refers to the prevention in whole or in part of the condition, or disease being prevented, including at least one symptom associated therewith or caused thereby.

Glycemic index value or glycemic index or GI refers to a number from 0 to 100 assigned to a food, with pure glucose arbitrarily given the value of 100, which represents the relative rise in the blood glucose level two hours after consuming that food. Nonlimiting exemplary GI of common sweeteners and sugars are provided in Table I. Low glycemic index foods have a GI of less than 50.

TABLE 1
Glycemic Index of Common Sweeteners and Sugars

| | Glycemic | | Glycemic |
|---|---|---|---|
| Sweetener | Index | Sweetener | Index |
| Maltodextrin | 110 | Glycerol | 5 |
| Maltose | 105 | Sorbitol | 4 |
| Dextrose | 100 | Lactitol | 3 |
| Glucose | 100 | Isomalt | 2 |
| Trehalose | 70 | Mannitol | 2 |
| HFCS-42 | 68 | Erythritol | 1 |
| Sucrose | 65 | Yacon Syrup | 1 |
| Caramel | 60 | Oligofructose | 1 |
| Golden Syrup | 60 | Inulin | 1 |
| Inverted Sugar | 60 | Brazzein | 0 |
| Refiners Syrup | 60 | Curculin | 0 |
| HFCS-55 | 58 | Glycyrrhizin | 0 |
| Blackstrap Molasses | 55 | Luo Han Guo | 0 |
| Maple Syrup | 54 | Miraculin | 0 |
| Honey | 50 | Monellin | 0 |
| Sorghum Syrup | 50 | Pentadin | 0 |
| Lactose | 45 | Stevia | 0 |
| Cane Juice | 43 | Thaumatin | 0 |
| Barley Malt Syrup | 42 | Acesulfame K | 0 |
| HSH | 35 | Alitame | 0 |
| Coconut Palm Sugar | 35 | Aspartame | 0 |
| Maltitol | 35 | Cyclamate | 0 |
| HFCS-90 | 31 | Neotame | 0 |
| Brown Rice Syrup | 25 | Saccharin | 0 |
| Fructose | 25 | Sucralose | 0 |
| Galactose | 25 | Glycerol | 5 |
| Agave Syrup | 15 | | |
| Xylitol | 12 | | |

Gel refers to chewable gel. Chewable gels can maintain their molded shape, can be elastic, and can yield to mastication. Chewable gels can be chewed before swallowing. The can have a short, tender, semi-elastic texture with good flavor release.

Essentially free refers to less than about 5 wt %, less than about 4 wt %, less than about 3 wt %, less than about 2 wt %, less than about 1 wt %, less than about 0.5 wt %, less than about 0.1 wt %, less than about 0.05 wt %, less than about 0.01 wt %, or free of detectable amounts of the named component.

The present technology provides a composition that includes pectin; and a rare sugar, polydextrose, or a combination thereof; wherein the composition is a gummy and essentially free of gelatin. The composition can have less than about 50 wt % sugar, a glycemic index of about 50 or less, about 10 calories or less, or a combination of two or more thereof. The composition is thus suitable for a wide range of patients or subjects including vegans, who often avoid products with gelatin, and those wishing to avoid compositions with significant amounts of simple sugars (e.g., sucrose and/or glucose) and/or high calorie compositions. These sugars not only contribute calories, but also have a high glycemic index (GI) value, which upon consumption can lead to a spike in glucose levels. Sugars with a high GI value are not tolerated well by people with metabolic disorders such as diabetes.

To that end, the composition can have less than about 50 wt % sugar. The composition can have less than about 45 wt %, less than about 40 wt %, less than about 35 wt %, less than about 30 wt %, less than about 25 wt %, less than about 20 wt %, less than about 15 wt %, less than about 10 wt %, or less than about 5 wt % sugar.

Similarly, the composition can have a glycemic index (GI) of about 50 or less. The composition can have a GI less than about 45, less than about 40, less than about 35, less than about 30, less than about 25, less than about 20, less than about 15, less than about 10, or less than about 5.

In the same vein, the composition can have about 10 calories or less. The composition can have about 10 calories, about 9 calories, about 8 calories, about 7 calories, about 6 calories, about 5 calories, about 4 calories, about 3 calories, about 2 calories, or about 1 calorie.

In a particular example, the composition can have less than about 50 wt % sugar and a glycemic index of about 50 or less. The composition can have less than about 50 wt % sugar and about 10 calories or less. The composition can have a glycemic index of about 50 or less and about 10 calories or less. The composition can have less than about 50 wt % sugar and about 10 calories or less. The composition can have less than about 50 wt % sugar, a glycemic index of about 50 or less, and about 10 calories or less.

The composition can include about 1 wt % to about 10 wt % of the pectin, which provides a composition with a suitable texture for making the composition into gummies More particularly, the composition can include about 2 wt % to about 5 wt % of the pectin including about 2.5 wt % to about 3.5 wt % or about 2.75 wt % to about 3.25 wt % of the pectin.

The composition can include the rare sugar. Rare sugars can exhibit a limited impact on increasing blood sugar and have a low glycemic index but have been shown to exhibit beneficial effects on controlling a patient or subject's weight. The composition can include the polydextrose. Polydextrose is a prebiotic that can have beneficial effects to a patient or subject's digestive system. The composition can include the rare sugar and the polydextrose.

The rare sugar can be a hexose. The rare sugar can include, but is not limited to, allulose (psicose), tagatose, sorbose, apiose, allose, altrose, melezitose, L-ribose, L-fucose, L-arabinose, D-turanose, D-leucrose, or a combination of two or more thereof. The rare sugar can include allulose, tagatose, sorbose, apiose, allose, altrose, melezitose, or a combination of two or more thereof. The rare sugar can include allulose, tagatose, sorbose, apiose, allose, or a combination of two or more thereof. The rare sugar can include allulose, tagatose, sorbose, allose, or a combination of two or more thereof. The rare sugar can include allulose. The rare sugar can consist of allulose.

The rare sugar can be a hydrolyzed disaccharide and/or hydrolyzed trisaccharide. For example, the rare sugar can be provided by the hydrolysis of a polysaccharide, oligosaccharide, trisaccharide (e.g., melezitose), and/or disaccharide (e.g., turanose). The rare sugar can be a hydrolyzed or partial hydrolyzed polysaccharide, oligosaccharide, trisaccharide, and/or disaccharide.

The composition can include about 5 wt % to about 75 wt % of the rare sugar. The composition can include about 10 wt % to about 70 wt % of the rare sugar including about 20 wt % to about 60 wt %, about 25 wt % to about 60 wt %, about 30 wt % to about 60 wt %, about 35 wt % to about 60 wt %, about 45 wt % to about 60 wt %, about 50 wt % to about 60 wt %, about 55 wt % to about 60 wt %, about 20 wt % to about 55 wt %, about 20 wt % to about 50 wt %, about 20 wt % to about 45 wt %, about 20 wt % to about 40 wt %, about 20 wt % to about 35 wt %, about 20 wt % to about 30 wt %, about 20 wt % to about 25 wt %, about 25 wt % to about 55 wt %, about 30 wt % to about 50 wt %, about 35 wt % to about 45 wt %, or about 45 wt % to about 55 wt % of the rare sugar.

The composition can include about 5 wt % to about 75 wt % of the polydextrose. The composition can include about 10 wt % to about 70 wt % of the polydextrose including about 10 wt % to about 60 wt %, about 20 wt % to about 60 wt %, about 25 wt % to about 60 wt %, about 30 wt % to about 60 wt %, about 35 wt % to about 60 wt %, about 45 wt % to about 60 wt %, about 50 wt % to about 60 wt %, about 55 wt % to about 60 wt %, 10 wt % to about 50 wt %, about 20 wt % to about 50 wt %, about 25 wt % to about 50 wt %, about 30 wt % to about 50 wt %, about 35 wt % to about 50 wt %, 10 wt % to about 40 wt %, about 20 wt % to about 40 wt %, about 25 wt % to about 40 wt %, about 30 wt % to about 40 wt %, about 35 wt % to about 40 wt %, 10 wt % to about 35 wt %, 15 wt % to about 35 wt %, about 20 wt % to about 35 wt %, about 25 wt % to about 35 wt %, about 30 wt % to about 35 wt %, about 20 wt % to about 55 wt %, about 20 wt % to about 50 wt %, about 20 wt % to about 45 wt %, about 20 wt % to about 40 wt %, about 20 wt % to about 35 wt %, about 20 wt % to about 30 wt %, about 20 wt % to about 25 wt %, about 25 wt % to about 55 wt %, about 30 wt % to about 50 wt %, about 35 wt % to about 45 wt %, or about 45 wt % to about 55 wt % of the polydextrose.

The polydextrose can be a polydextrose composition that includes at least about 80 wt %, at least about 85 wt %, or at least 90 wt % polydextrose. The polydextrose composition can include less than about 10 wt %, less than about 9 wt %, less than about 8 wt %, or less than about 7 wt % glucose/sorbitol. The polydextrose composition can include less than about 10 wt %, less than about 8 wt %, less than about 6 wt %, or less than about 5 wt % dextrose. The polydextrose composition can include less than about 10 wt %, less than about 8 wt %, less than about 6 wt %, or less than about 3 wt % sorbitol. The polydextrose can be vegan. The polydextrose can exhibit a limited impact on increasing blood sugar. The polydextrose can have a low glycemic index. The polydextrose can be a non-thickening buking agent. The polydextrose can be serve as a prebiotic fiber. The polydextrose can be resistant to hydrolysis. The polydextrose can be substituted with oligo and/or polysaccharide(s) that do not contribute glucose or dextrose to the composition. The polydextrose can be partially or fully substituted. Nonlimiting exemplary oligo and polysaccharides include hemi-cellulose, cellulose, corn, potato or tapioca modified starches, resistant starch, resistant dextrin, or a combination of two or more thereof.

The composition can include about 15 wt % to about 35 wt % of the polydextrose (including about 20 wt % to about 30 wt %), about 1 wt % to about 5 wt % of the pectin (including about 2 wt % to about 4 wt %), and about 30 wt % to about 60 wt % of the rare sugar (e.g., allulose) (including about 40 wt % to about 55 wt %). The composition can include about 20 wt % to about 30 wt % of the polydextrose, about 2 wt % to about 4 wt % of the pectin, and about 40 wt % to about 55 wt % of the rare sugar (e.g., allulose).

The polydextrose and rare sugar can have a weight ratio range of about 10:1 to about 1:10 (including about 4:1 to about 1:6). The polydextrose and rare sugar can have a weight ratio range of about 1:1 to about 1:4 (including about 1:1 to about 1:2). The polydextrose and pectin can have a weight ratio range of about 20:1 to about 1:1. The polydextrose and pectin can have a weight ratio range of about 15:1 to about 5:1. The rare sugar and pectin can have a weight ratio range of about 30:1 to about 5:1. The rare sugar and pectin can have a weight ratio range of about 20:1 to about 10:1.

The composition can include less than about 10 wt % of high potency sweeteners. This allows the high potency sweeteners to improve the taste of the composition by making it sweet, however, the high potency sweetener does not significantly add calories to the composition. The composition can include less than about 5 wt %, less than about 4 wt %, less than about 3 wt %, less than about 2 wt %, less than about 1 wt %, or less than about 0.5 wt % of high potency sweeteners. The composition can be essentially free of high potency sweeteners. The composition can be free of high potency sweeteners.

The composition can include less than about 10 wt % of monosaccharides, wherein the monosaccharide is not a rare sugar. The composition can include less than about 5 wt %, less than about 4 wt %, less than about 3 wt %, less than about 2 wt %, less than about 1 wt %, or less than about 0.5 wt % of monosaccharides, wherein the monosaccharide is not a rare sugar. The composition can be essentially free of monosaccharides, wherein the monosaccharide is not a rare sugar. The composition can be free of monosaccharides, wherein the monosaccharide is not a rare sugar.

The composition can include less than about 10 wt % of disaccharides, wherein the disaccharide is not a rare sugar or precursor thereof. The composition can include less than about 5 wt %, less than about 4 wt %, less than about 3 wt %, less than about 2 wt %, less than about 1 wt %, or less than about 0.5 wt % of disaccharides, wherein the disaccharide is not a rare sugar or precursor thereof. The composition can be essentially free of disaccharides, wherein the disaccharide is not a rare sugar or precursor thereof. The composition can be free of disaccharides, wherein the disaccharide is not a rare sugar or precursor thereof.

The composition can include less than about 10 wt % of oligosaccharides and/or polysaccharides, wherein the oligosaccharide and/or the polysaccharide does not include polydextrose, inulin, inulin-type fructans, resistant starch, resistant dextrin, or combinations to two or more thereof. The composition can include less than about 5 wt %, less than about 4 wt %, less than about 3 wt %, less than about 2 wt %, less than about 1 wt %, or less than about 0.5 wt % of oligosaccharides and/or polysaccharides, wherein the oligosaccharide and/or the polysaccharide does not include polydextrose, inulin, inulin-type fructans, resistant starch, resistant dextrin, or combinations to two or more thereof. The composition can be essentially free of oligosaccharides and/or polysaccharides, wherein the oligosaccharide and/or the polysaccharide does not include polydextrose, inulin, inulin-type fructans, resistant starch, resistant dextrin, or combinations to two or more thereof. The composition can be free of oligosaccharides and/or polysaccharides, wherein the oligosaccharide and/or the polysaccharide does not include polydextrose, inulin, inulin-type fructans, resistant starch, resistant dextrin, or combinations to two or more thereof.

The composition can include less than about 10 wt % of sugar alcohols. The composition can include less than about 5 wt %, less than about 4 wt %, less than about 3 wt %, less than about 2 wt %, less than about 1 wt %, or less than about 0.5 wt % of sugar alcohols. The composition can be essentially free of sugar alcohols. The composition can be free of sugar alcohols.

The composition can include less than about 10 wt % of natural sugars. The composition can include less than about 5 wt %, less than about 4 wt %, less than about 3 wt %, less than about 2 wt %, less than about 1 wt %, or less than about 0.5 wt % of natural sugars. The composition can be essentially free of natural sugars. The composition can be free of natural sugars.

The composition can include less than about 10 wt % of artificial sugars. The composition can include less than about 5 wt %, less than about 4 wt %, less than about 3 wt %, less than about 2 wt %, less than about 1 wt %, or less than about 0.5 wt % of artificial sugars. The composition can be essentially free of artificial sugars. The composition can be free of artificial sugars.

The composition can include less than about 10 wt % of trehalose. The composition can include less than about 5 wt %, less than about 4 wt %, less than about 3 wt %, less than about 2 wt %, less than about 1 wt %, or less than about 0.5 wt % of trehalose. The composition can be essentially free of trehalose. The composition can be free of trehalose.

The composition can include less than about 10 wt % of isomaltulose. The composition can include less than about 5 wt %, less than about 4 wt %, less than about 3 wt %, less than about 2 wt %, less than about 1 wt %, or less than about 0.5 wt % of isomaltulose. The composition can be essentially free of isomaltulose. The composition can be free of isomaltulose.

The composition can include less than about 10 wt % of tagatose. The composition can include less than about 5 wt %, less than about 4 wt %, less than about 3 wt %, less than about 2 wt %, less than about 1 wt %, or less than about 0.5 wt % of tagatose. The composition can be essentially free of tagatose. The composition can be free of tagatose.

The composition can include less than about 10 wt % of any component with a glycemic index above about 15. The composition can include less than about 5 wt %, less than about 4 wt %, less than about 3 wt %, less than about 2 wt %, less than about 1 wt %, or less than about 0.5 wt % of any component with a glycemic index above about 15. The composition can be essentially free of any component with a glycemic index above about 15. The composition can be free of any component with a glycemic index above about 15.

The composition can include less than about 10 wt % of corn syrup, high fructose corn syrup, glucose syrup, isomaltoligosaccharide syrup, agave syrup, invert syrup, high maltose syrup, rice syrup, tapioca syrup, sugar cane, or a combination of two or more thereof. The composition can include less than about 5 wt %, less than about 4 wt %, less than about 3 wt %, less than about 2 wt %, less than about 1 wt %, or less than about 0.5 wt % of corn syrup, high fructose corn syrup, glucose syrup, isomaltoligosaccharide syrup, agave syrup, invert syrup, high maltose syrup, rice syrup, tapioca syrup, sugar cane, or a combination of two or more thereof. The composition can be essentially free of corn syrup, high fructose corn syrup, glucose syrup, isomaltoligosaccharide syrup, agave syrup, invert syrup, high maltose syrup, rice syrup, tapioca syrup, sugar cane, or a combination of two or more thereof. The composition can be free of corn syrup, high fructose corn syrup, glucose syrup, isomaltoligosaccharide syrup, agave syrup, invert syrup, high maltose syrup, rice syrup, tapioca syrup, sugar cane, or a combination of two or more thereof.

The composition can include a vitamin, mineral, green blend, vegetable blend, fruit blend, probiotic culture, antioxidant, fiber blend, or a combination of two or more thereof. The composition can include one or more vitamins (e.g., vitamin A, vitamin C, vitamin D, vitamin B6, vitamin B12, vitamin E, vitamin K1, vitamin K2, vitamin K3, folate, thiamin, niacin, pantothenic acid, omega-3 fatty acid, or a combination of two or more thereof). The composition can include one or more minerals (e.g., iron, zinc, magnesium, calcium, or a combination of two or more thereof). The composition can include a green blend (e.g., artichoke leaf extract, blue-green algae (e.g., spirulina), aloe vera, kale, moringa leaf, broccoli, barley grass, chlorella, parsley, spinach, celery, wheat grass, alfalfa, carrot root, beet root, dulse, oat grass, cabbage, or a combination of two or more thereof). The green blend can be in powder and/or liquid form. The composition can include a vegetable blend (e.g., broccoli, spinach, carrots, beets, peas, tomatoes, cabbage, cauliflower, pumpkin, asparagus, brussel sprouts, cucumber, or a combination of two or more thereof). The composition can include a fruit blend (e.g., apple, blueberry, raspberry, noni, goji berry, amla, acai, pineapple, rose hips, monk, acerola, pomegranate, banana, strawberry, or a combination of two or more thereof). The composition can include probiotic culture (e.g., Bacillus subtilis, Bacillus coagulans, Lactobacillus rhamnosus, Lactobacillus paracesei, Lactobacillus acidophilus, Lactobacillus plantarum, Bifidobacterium breve, Bifidobacterium longum, Bifidobacterium adolescentis, Bifidobacterium lactis, Bifidobacterium bifidum, or a combination of two or more thereof). The composition can include one or more antioxidants (e.g., acai extract (Euterpe oleracea), goji berry extract (Lycium barbarum), noni extract, mangosteen fruit, pomegranate fruit extract, trans-Resveratrol, raspberry fruit powder, black cherry fruit powder, blueberry fruit powder, elderberry extract, grape skin extract, antioxidant vitamin(s), glutathione, lipoic acid, uric acid, carotenes, ubiquinol, antioxidant enzymes, food preservative antioxidant(s), or a combination of two or more thereof). The composition can include a fiber blend (e.g., oat fiber, brown rice bran, apple pectin, citrus pectin, soy fiber, psyllium husk powder, or a combination of two more thereof). Other examples include Angelica sinesis, Astragalus, red dates, goji berry, longan berry, Condonoposis pilosula (Dangshen), ginseng, gandoerma (Linzhi), coix seed, ginger, Bletilla tuber (Baiji), white Atractylodes rhizome (Baishu), Radix angelicae Dahuricae (Baizhi), Typhonium tuber (baifuzi), Radix ampelopsis (bailian), Rhizoma bletillae (baiji), Rhizoma polygonati odorati (yuzhu), Adenophora root (shashen), lily bulb (baihe), mune bean, licorice, ampelopsis root (bailian), white peony root (baishao), Chinese Yam (Rhizoma dioscoreae, shanyao), poria (fulin), Schisandra, mint, aloe, lemon peel, lemon, orange peel, Polygonum multiflorum (he-shou-wu), ginko, turmeric, gotu kola (Centella asiatica), ashwagandha, Tribulus terrestris, mucunna pruriens, guarana, mate (Ilex paraguariensis), Ephedra (Ephedra sinica or ma-huang), ciwujia, rose, lotus, honeysuckle, chrysanthemum, Osmanthus fragran, jasmine, dandelion, peach blossom, blueberry, acai berry, raspberry, blackberry, huckleberry, cranberry, strawberry, lingonberry, gooseberry, black or red currants, cloudberry, hawthorn berry, coffee, moca, or extracts or isolates thereof. The composition can include about 0.1 wt % to about 50 wt % of the vitamin, mineral, green blend, vegetable blend, fruit blend, probiotic culture, antioxidant, and/or fiber blend. The composition can include about 1 wt % to about 40 wt % of the vitamin, mineral, green blend, vegetable blend, fruit blend, probiotic culture, antioxidant, and/or fiber blend. The composition can include about 5 wt % to about 30 wt % of the vitamin, mineral, green blend, vegetable blend, fruit blend, probiotic culture, antioxidant, and/or fiber blend. The composition can include about 5 wt % to about 20 wt % of the vitamin, mineral, green blend, vegetable blend, fruit blend, probiotic culture, antioxidant, and/or fiber blend. The composition can include about 0.5 wt % to about 10 wt % of the vitamin, mineral, green blend, vegetable blend, fruit blend, probiotic culture, antioxidant, and/or fiber blend. The composition can include about 0.5 wt % to about 5 wt % of the vitamin, mineral, green blend, vegetable blend, fruit blend, probiotic culture, antioxidant, and/or fiber blend.

The composition can include an active ingredient. Nonlimiting exemplary active ingredients include an antibacterial agent, an antifungal agent, a pain or fever reliever, an analgesics, an anti-inflammatory agent, a steroid, a diabetic medication, an antidiarrheal agent, an antiulcer agent, a proton pump inhibitor, a laxative or cathartic agent, a diuretic agent, an antacid, an antihistamine, a decongestant, an antitussive or expectorant agent, a cough suppressant, a cold medicine, a motion sickness medication, a medication for treating alcoholism or opioid dependence, a smoke cessation agent, an anti-anxiety agent, an antidepressant, a mood stabilizer, an antipsychotic agent, a stimulant, a benzodiazepine, an anxiolytic agent, a Z-drug, a melatonergic agent, a barbiturate, an antidepressant, an antipsychotic agent, a cardiovascular agent, an anti-cancer agent, an immune suppressant, a blood thinner, acetaminophen, aspirin, salicylic acid, ibuprofen, naproxen, cetirizine, diphenhydramine, loratadine, chlorpheniramine, brompheniramine, alimemazine, cyprohetadine, doxylamine, hydroxyzine, promethazine, caffeine, nicotine, melatonin, or a combination of two or more thereof. The active ingredient can include apple cider vinegar, melatonin, caffeine, cannabis extract (e.g., hemp extract and/or cannabidiol, cannabinol, cannabigerol), ashwagandha extract, whole root powder, acetaminophen, ibuprofen, botanicals (powder and/or extract), hyaluronic acid, amino acids (e.g., theanine and/or gamma-aminobutyric acid), peptides, ceramide, collagen, keratin, biotin, lycopene, or a combination of two or more thereof. The composition can include about 0.1 wt % to about 40 wt % of the active ingredient. The composition can include about 0.5 wt % to about 30 wt % of the active ingredient. The composition can include about 0.5 wt % to about 20 wt % of the active ingredient. The composition can include about 0.5 wt % to about 10 wt % of the active ingredient. The composition can include about 0.5 wt % to about 5 wt % of the active ingredient.

The composition can include a gelling agent different from the pectin. The gelling agent can include agar, gum, high molecular weight carboxymethylcellulose, alginate, starch (including modified food starch), or a combination of two or more thereof.

The composition can include water. The composition can include less than about 35 wt % water. The composition can include about 5 wt % to about 30 wt % water or about 5 wt % to about 25 wt % water. The composition can include less than about 20 wt % water. The composition can include about 5 wt % to about 20 wt % water or about 11 wt % to about 14 wt % water.

The composition can include an emulsifier such as lecithin. The composition can include about 0.1 wt % to about 10 wt % of the emulsifying agent. The composition can include about 0.1 wt % to about 5 wt % of the emulsifying agent.

The composition can include an edible carboxylic acid or a salt thereof (e.g., malic acid, citric acid, or a combination thereof. The composition can include about 0.1 wt % to about 5 wt % (including about 1 wt % to about 3 wt %) edible carboxylic acid or a salt thereof.

The composition can include flavoring agent. The composition can include about wt % to about 1 wt % (including about 0.1 wt % to about 1 wt %) of the flavoring agent.

The composition can be provided in any desired size and shape. Each gummy can be about 1 g to about 20 g. Each gummy can be about 1 g to about 10 g (e.g., about 1, 2, 3, 4, 5, 6, 7, 8, 9, or 10 g). Each gummy can be about 2 g to about 4 g. Each gummy can have less than about 10 calories (e.g., about 1, 2, 3, 4, 5, 6, 7, 8, 9, or 10 calories). Each gummy can have about 1 calorie to about 5 calories. For example, each gummy can have less than about 10 calories and can be about 1 g to about 10 g or each gummy can have about 1 calorie to about 5 calories and can be about 2 g to about 4 g.

The composition can be in a single homogenous layer.

The composition can be soluble in an aqueous media. The composition can be orally soluble.

The composition can exhibit a similar sweetness profile as sucrose. The composition can be free of a metallic taste when the composition includes mineral(s) and/or green blend and/or vegetable blend.

The composition can be a gel structure. The composition can be firm but resilient. The composition can be clear. The composition can be opaque. The composition can be substantially crystal free. The composition can have good flavor release.

Under ambient conditions, the composition can have a shelf-life and be essentially free of crystallization for about 1 month to about 5 years (including about 6 months to about 3 years). The composition can be essentially free of syneresis except when applied into a yogurt or jam type of product.

In another aspect, the present technology provides a method for treating a patient with a nutritional deficiency or preventing a nutritional deficiency. The method includes administering at least one gummy composition provided herein. The method can include administering one or two gummy compositions daily. Each gummy can include about 25% or more of the daily recommended amount of the any of the components disclosed herein. Each gummy can include about 25% to about 100% or more of the daily recommended amount of the any of the components disclosed herein.

### EXAMPLES

The present technology is further illustrated by the following examples, which should not be construed as limiting in any way.

### Example 1: A Process for Making a Plurality of Gummies

Pectin and water were added together under mixing and heated. To the heated pectin composition, allulose and polydextrose were added under mixing. The mixture was cooled to room temperature followed by addition of a hydrated mixture of botanicals, vitamins, minerals, probiotics, and active ingredients. Lecithin was then added, followed by malic acid, citric acid, and a natural flavor. The mixture was heated in an agitator (85-115° C.) for about 5 to 90 minutes. The heated composition was poured into molds and allowed to cool. A plurality of gummies were then removed from the molds.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A composition comprising:
pectin; and
a rare sugar, polydextrose, or a combination thereof; wherein:
the composition is a gummy;
the composition has less than about 50 wt % sugar, a glycemic index of about 50 or less, about 10 calories or less, or a combination of two or more thereof; and
the composition is essentially free of gelatin.

2. The composition of claim 1, wherein the composition has about 10 calories or less.

3. The composition of claim 1, wherein the composition comprises the rare sugar.

4. The composition of claim 3, wherein the rare sugar is a hexose.

5. The composition of claim 3, wherein the rare sugar is selected from the group consisting of allulose, tagatose, sorbose, allose, or a combination of two or more thereof.

6. The composition of claim 5, wherein the rare sugar comprises allulose.

7. The composition of claim 3, wherein the rare sugar is one of a hydrolyzed disaccharide and a hydrolyzed trisaccharide.

8. The composition of claim 3, wherein the composition comprises about 5 wt % to about 75 wt % of the rare sugar.

9. The composition of claim 8, wherein the composition comprises about 20 wt % to about 60 wt % of the rare sugar.

10. The composition of claim 1, wherein the composition comprises the polydextrose.

11. The composition of claim 10, wherein the composition comprises about 5 wt % to about 75 wt % of the polydextrose.

12. The composition of claim 11, wherein the composition comprises about 10 wt % to about 40 wt % of the polydextrose.

13. The composition of claim 1, wherein the composition comprises about 2 wt % to about 5 wt % of the pectin.

14. The composition of claim 1, wherein the composition is essentially free of high potency sweeteners.

15. The composition of claim 1, wherein the composition is essentially free of monosaccharides, wherein the monosaccharide is not a rare sugar.

16. The composition of claim 1, wherein the composition is essentially free of disaccharides, wherein the disaccharide is not a rare sugar or precursor thereof.

17. The composition of claim 1, wherein the composition is essentially free of oligosaccharides or polysaccharides, wherein the oligosaccharide or the polysaccharide does not include polydextrose, inulin, inulin-type fructans, resistant starch, or resistant dextrin.

18. The composition of claim 1, wherein the composition is essentially free of sugar alcohols.

19. The composition of claim 1, wherein the composition is essentially free of natural sugars.

20. The composition of claim 1, wherein the composition is essentially free of any component with a glycemic index above about 15.

21. The composition of claim 1, wherein the composition is essentially free of corn syrup, high fructose corn syrup, glucose syrup, isomaltoligosaccharide syrup, agave syrup, invert syrup, high maltose syrup, rice syrup, tapioca syrup, sugar cane, or a combination of two or more thereof.

22. The composition of claim 1, further comprising a mineral, wherein the mineral comprises iron, zinc, magnesium, calcium, or a combination of two or more thereof.

23. The composition of claim 1, further comprising a gelling agent different from the pectin.

24. The composition of claim 23, wherein the gelling agent comprises agar, gum, high molecular weight carboxymethylcellulose, alginate, starch (including modified food starch), or a combination of two or more thereof.

25. The composition of claim 1, wherein the composition comprises less than about 35 wt % water.

26. The composition of claim 1, further comprising an active ingredient, wherein the active ingredient comprises apple cider vinegar, melatonin, caffeine, cannabis extract (e.g., hemp extract, cannabidiol, cannabinol, and cannabigerol), ashwagandha extract, whole root powder, acetaminophen, ibuprofen, botanicals (powder or extract), hyaluronic acid, amino acids (e.g., theanine or gamma-aminobutyric acid), peptides, ceramide, collagen, keratin, biotin, lycopene, or a combination of two or more thereof.

27. The composition of claim 1, wherein each gummy is about 1 g to about 10 g.

28. The composition of claim 27, wherein each gummy has less than about 10 calories.

29. The composition of claim 1, wherein the composition is a single homogenous layer.

30. The composition of claim 1, wherein the composition is soluble in an aqueous media.

31. The composition of claim 1, wherein the composition is a gel.

32. The composition of claim 1, wherein the composition is substantially crystal free.
